(19) United States
(12) Patent Application Publication
(10) Pub. No.: US 2021/0076950 A1
(43) Pub. Date: Mar. 18, 2021

(54) Health and Wellness Monitoring System for Multiple Users

(71) Applicant: Hall Labs LLC, Provo, UT (US)

(72) Inventors: David R. Hall, Provo, UT (US); Vivek Garg, Murray, UT (US); Steven Lund, Provo, UT (US); Monte Johnson, Provo, UT (US); Jeff Duncan, Provo, UT (US); Kelly Koch, Chicago, UT (US)

(73) Assignee: Hall Labs LLC, Provo, UT (US)

(21) Appl. No.: 16/572,586

(22) Filed: Sep. 16, 2019

(51) Int. Cl. A61B 5/0205; G16H 40/67; G16H 15/00; G16H 10/40; A61B 10/00; A61B 5/20; A61B 5/02; A61B 5/01; A61B 5/103; A61B 5/00; A61B 5/117

(52) U.S. Cl. CPC A61B 5/02055; A61B 5/024; G16H 15/00; G16H 10/40; A61B 10/0038; A61B 5/208; A61B 5/02042; A61B 5/01; A61B 5/1032; A61B 5/441; A61B 5/7275; A61B 5/746; A61B 5/0022; A61B 5/6887; A61B 5/486; A61B 5/117; A61B 2010/0083; G16H 40/67

(57) ABSTRACT

The invention is a system for monitoring and reporting health and wellness data. The system includes a pre-qualification mechanism, whereby an individual can become a pre-qualified user. The system also includes a plurality of stations, with each station including a user identification unit to determine if the user is a pre-qualified user and, if so, identify that pre-qualified user and a toilet. The toilet includes a bowl to receive excreta, two or more sensors configured to detect properties of the excreta from a pre-qualified user, and a bowl clearing mechanism to dispose of the excreta. Each station also includes a station processor configured to receive signals from the two or more sensors and from the user identification unit to create excreta event data for the identified pre-qualified user. Each station also includes a station data communication unit. The system also includes a central base that, in turn, includes a central base data receiving unit configured to receive excreta event data via the station data communication unit from each of the plurality of stations. The central base also includes a central base data storage unit configured to store excreta event data. The central base further includes a central base data processor configured to process the excreta event data for each pre-qualified user and produce health and wellness data for that pre-qualified user. The central base still further includes a central base data reporting unit configured to report health and wellness data to the appropriate pre-qualified user.

### TECHNICAL FIELD

This invention relates to health and wellness monitoring.

### BACKGROUND

Data from excreta is used by doctors and individuals to track health and wellness in a plethora of ways. The frequency of collecting such data varies from annual wellness checkups to daily monitoring of certain health conditions. However, innovations in the field of smart toilets has made health and wellness tracking far more accessible. Sample collection can now be done by a smart toilet, without any additional time spent by the user, or a health aid. Also, sensors in the toilet can collect information that, with processing, can inform the user about many different wellness markers, such as hydration levels, the presence of ketones, levels of certain vitamins, the amount of fat consumption in the diet, and many other things. Additionally, the smart toilet provides an enhancement to other excreta testing in that it can be linked to the user's other data and provide the user with information on comparative trends in their results.

Just a few examples of smart toilets can be seen in the following U.S. Patents and Published Applications: U.S. Pat. No. 9,867,513, entitled “MEDICAL TOILET WITH USER AUTHENTICATION”; U.S. Pat. No. 10,123,784, entitled “IN SITU SPECIMEN COLLECTION RECEPTACLE IN A TOILET AND BEING IN COMMUNICATION WITH A SPECTRAL ANALYZER”; U.S. Pat. No. 10,273,674, entitled “TOILET BOWL FOR SEPARATING FECAL MATTER AND URINE FOR COLLECTION AND ANALYSIS”; US 2016/0000378, entitled “HUMAN HEALTH PROPERTY MONITORING SYSTEM”; US 2018/0020984, entitled “METHOD OF MONITORING HEALTH WHILE USING A TOILET”; US 2018/0055488, entitled “TOILET VOLATILE ORGANIC COMPOUND ANALYSIS SYSTEM FOR URINE”; US 2018/0078191, entitled “MEDICAL TOILET FOR COLLECTING AND ANALYZING MULTIPLE METRICS”; US 2018/0140284, entitled “MEDICAL TOILET WITH USER CUSTOMIZED HEALTH METRIC VALIDATION SYSTEM”; US 2018/0165417, entitled “BATHROOM TELEMEDICINE STATION.” The disclosures of all of these patents and applications are incorporated by reference.

However, the myriad wellness benefits promised by smart toilets remain, as yet, limited in their impact. One reason for this is the issue of accessibility, owing to the typical high upfront cost of one unit. Another reason is lack of awareness, i.e. not enough people know about the availability and benefits of smart toilets.

### SUMMARY

In one aspect, the invention is a system for monitoring and reporting health and wellness data. The system includes a pre-qualification mechanism, whereby an individual can become a pre-qualified user. The system also includes a plurality of stations, with each station including a user identification unit to determine if the user is a pre-qualified user and, if so, identify that pre-qualified user and a toilet. The toilet includes a bowl to receive excreta, two or more sensors configured to detect properties of the excreta from a pre-qualified user, and a bowl clearing mechanism to dispose of the excreta. Each station also includes a station processor configured to receive signals from the two or more sensors and from the user identification unit to create excreta event data for the identified pre-qualified user. Each station also includes a station data communication unit. The system also includes a central base that, in turn, includes a central base data receiving unit configured to receive excreta event data via the station data communication unit from each of the plurality of stations. The central base also includes a central base data storage unit configured to store excreta event data. The central base further includes a central base data processor configured to process the excreta event data for each pre-qualified user and produce health and wellness data for that pre-qualified user. The central base still further includes a central base data reporting unit configured to report health and wellness data to the appropriate pre-qualified user.

### BRIEF DESCRIPTION OF THE DRAWINGS

The following drawings are provided to illustrate certain embodiments described herein. The drawings are merely illustrative and are not intended to limit the scope of claimed inventions and are not intended to show every potential feature or embodiment of the claimed inventions. The drawings are not necessarily drawn to scale; in some instances, certain elements of the drawing may be enlarged with respect to other elements of the drawing for purposes of illustration.

FIG. 1 is an illustration of a station, including user, user identification unit, and smart toilet.

FIG. 2 is an illustration depicting a station with an optically readable method of user verification.

FIG. 3 depicts an optically readable user identification code, displayed on a smart device and paper.

FIG. 4 is an illustration of a QR code on the station that is scanned by a user's smart device.

FIG. 5 is an illustration depicting a station with a magnetically readable method of user identification.

FIG. 6 is an illustration depicting a station with a biometric method of user identification.

FIG. 7 is an illustration of a touchpad on the station.

FIG. 8 is a cross sectional view of the smart toilet.

FIG. 9 is a perspective view of a smart toilet, including attached non-excreta sensors.

FIG. 10 is a depiction of data transmission connectivity for a station, central base station, and a user device.

FIG. 11 is an illustration of a station featuring the station processor, sample user identification unit, and smart toilet, depicting points of data transmission and reception.

FIG. 12 shows a printout of health and wellness data for a user, with accompanying instructions.

FIG. 13 shows one feature of the user app running on a smart device.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

As used herein, “smart toilet” is meant to refer to a fixture that functions as a traditional toilet and is equipped with other features. Although in general parlance a “smart” device implies internet connectivity, for our sake the term “smart toilet” also implies that the toilet is equipped with other features, including, at a minimum, health and wellness data collection capabilities.

As used herein, “user identification unit” refers to a feature of the station, which has the purpose of identifying pre-qualified users.

The purpose of the invention is to provide a network in which individuals may access the benefits of smart toilets, while larger entities, from fitness centers to health insurers, are incentivized to provide access to stations containing these smart toilets. Different methods of access are encompassed within the invention, and all are designed to offer the full benefits including the tracking of trends in health and wellness data, and accompanying smart-device enabled apps.

The smart toilet that can collect health and wellness data offers much promise to different groups. Most individuals are at least somewhat interested in cultivating their wellness. Monitoring health indicators can be a convenient way to verify the effectiveness of one's diet, fitness, or stress management plans. For example, individuals on a restricted diet may be vitamin deficient, while fitness training can lead to dehydration. Regular tracking of myriad health markers is not only beneficial for the unwell, but is of interest to the general population. Additionally, there are certain groups that need to monitor their personal data more carefully. For example, diabetics know that ketones in the urine can signal dangerous insulin insufficiency. However, performing this additional test daily would be burdensome, and so is reserved for when the diabetic is feeling unwell. Thus, access to a smart toilet will aid diabetics in managing their health and may also give them peace of mind.

This is only one example of a specific use met by the smart toilet. The information collected from a smart toilet and its affiliated devices can provide information concerning a person's nutritional needs, cardiac health, metabolic functioning, and more. What is more, the data from a single event can be compared, given gender, age, race, and other demographics, to detect any indicated outliers against norms, i.e. predetermined standards. These predetermined standards will vary by age, gender, and race, when appropriate, and identified to the user as the “Healthy Range” in health and wellness reports. There are different ways that “healthy” and “normal” ranges are determined, and some predetermined standards are the same across all individuals. Also, the health and wellness report from one event may be compared against the user's history to observe trends. As just one example, the presence of certain proteins in the urine can be an indicator for disease risk before any symptoms are present. Consequently, while the smart toilet may appeal most to those looking to monitor existing known health conditions or following a diet and fitness routine, it can benefit anyone.

One benefit of having access to trends in health and wellness information is it gives the user the ability to monitor certain known risk factors, as well as to track the efficacy of lifestyle or drug interventions on other health markers. For example, increased cardiovascular fitness is generally correlated to a lower resting heart rate. However, one's resting heart rate can be difficult to accurately assess from only one reading. Also, resting heart rate is something that takes time to be affected by an exercise regime. This is one way that tracking trends in a user's health and wellness information provides more benefits than a single reading is able to, as an average of multiple readings over time provides a much more reliable indicator of an individual's resting heart rate, and any gradual changes to it.

Smart devices, i.e. smart phones or tablet computers, and other devices designed to sync up with them, are being used to track all sorts of wellness information. Examples include wearable fitness trackers, digital food diaries, internet-connected scales, and under-mattress sleep trackers. People are taking an active role in their health and a big part of that is gathering and monitoring data. The smart toilet can provide people with a more in-depth level of health tracking than other devices available to consumers.

Despite the interest in wellness and data tracking, access to a smart toilet is only available to a limited number of people. One reason for this is the relative high cost of the device. The present invention provides a system in which individual users may have access to a network of smart toilets that larger entities own or lease, through a variety of possible routes.

In accordance with the invention, the units may be located in a public park, a doctor's office, or a fitness center. One object of the invention is to provide a way for many users to have access to a smart toilet by dispersing the high price. Another object is to increase awareness of the availability of smart toilets and their benefits.

In the system, there is a minimum of two stations. A station includes a number of things, primarily a smart toilet.

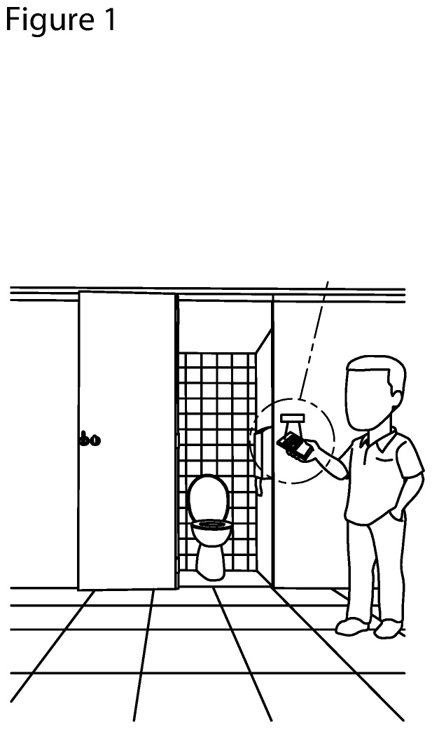

FIG. 1 depicts one example of a station. The system includes a pre-qualification mechanism, through which an individual becomes a pre-qualified user. In FIG. 1, the user is accessing the pre-qualification mechanism via his smart device, such as his smart phone 102. In other systems, the pre-qualification unit may be a stand-alone device adjacent to the station, and in yet another embodiment, the pre-qualification mechanism may be a separate feature built into the user identification unit. The different methods for pre-qualification will be discussed further.

One method of access is a subscription model, in which the user is qualified for an unlimited number of visits for a prepaid term. Another is a membership model, in which a user pre-qualifies for a certain number of visits. Another method for pre-qualification is an on-site purchase, the advantage of which is that no membership may be required, e.g. for the technology shy. These pre-qualification methods may be on a smart phone connected to the internet, as shown in FIG. 1. A user may become pre-qualified through a dedicated app, or signing up through a generally accessible website. The above embodiments, including a stand-alone pre-qualification device or one that is physically integrated with the user identification unit, are possible methods for on-site purchase of qualification. Pre-qualification, coupled with user identification, grants access. As such, “access” will be used to describe both methods of pre-qualification and user identification.

Each station includes a user identification unit, as seen in FIG. 1. The purpose of the user identification is twofold: (1) to verify that the individual attempting to gain access to the smart toilet is a qualified user, and (2) to link the health and wellness data received to their account, if applicable. The user identification method depicted in FIG. 1 makes use of Bluetooth technology. A pre-qualified user can pre-install an app on their smart device 102 that is able to connect to the user identification unit 104 via a Bluetooth network and identify itself, and thus the user's identity.

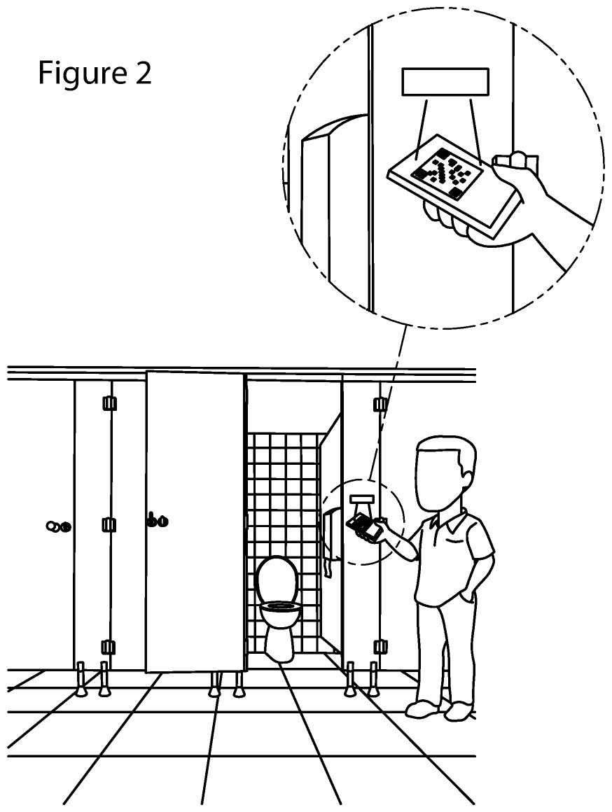

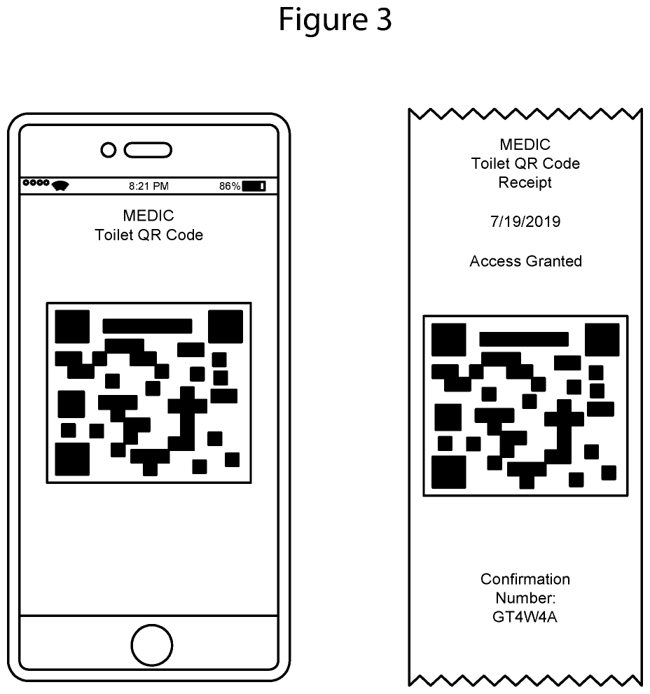

As seen in FIG. 2, another example of a user ID unit works by optically reading a unique code. A preferred embodiment of this is typically referred to as QR code. An optical reading user ID unit is the preferred embodiment. The user ID unit in FIG. 2 is equipped with an optical reader 202. This optical reader is scanning the QR code 204 unique to the user. As seen in FIG. 3, a QR code 302 is easily stored and producible on a smart phone 304, either in an app or as a digital photo. Alternatively, a QR code may also be printed out on-site. Print-out 306 depicts a QR code that is printed on-site at the station. A printed-out QR code is one way that users who choose on-site purchase may gain access to a station. In another alternative, a user who is already pre-qualified is able to print out the relevant QR code, in the case that they do not have their smart device but would like to use a station. Still another alternative is to issue membership cards with QR codes, magnetic strips or some other machine-readable identification built in.

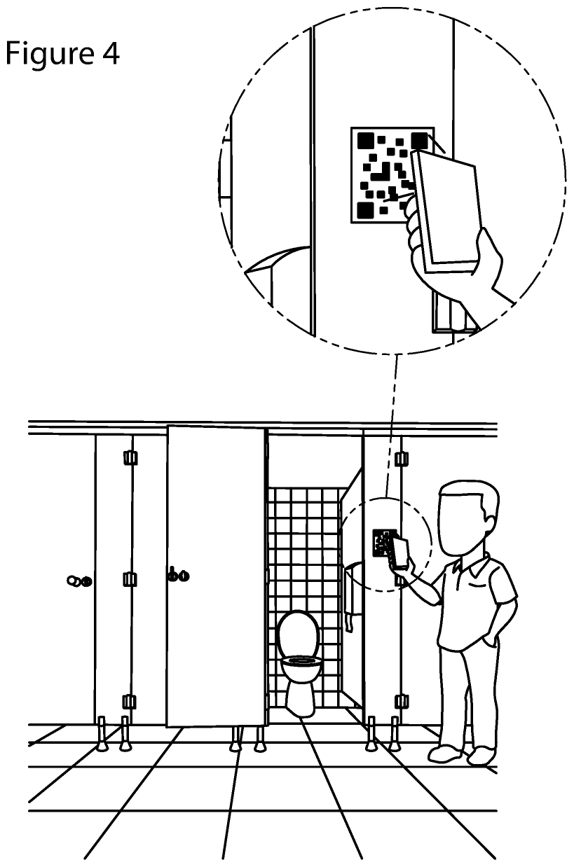

While the preferred mode is for a QR or similar code to be scanned by the user ID unit, an alternative, depicted in FIG. 4, is for the user ID unit to have a code to be scanned by the user's smart device, e.g. smart phone. In this embodiment shown in FIG. 4, the user first scans a unique code 404 for that particular station with their smart phone 402. That station identifier is then transmitted to a central base and, because the user is pre-qualified, i.e. through an app, the central base informs the user ID unit at that station that the user is pre-qualified and grants that user access.

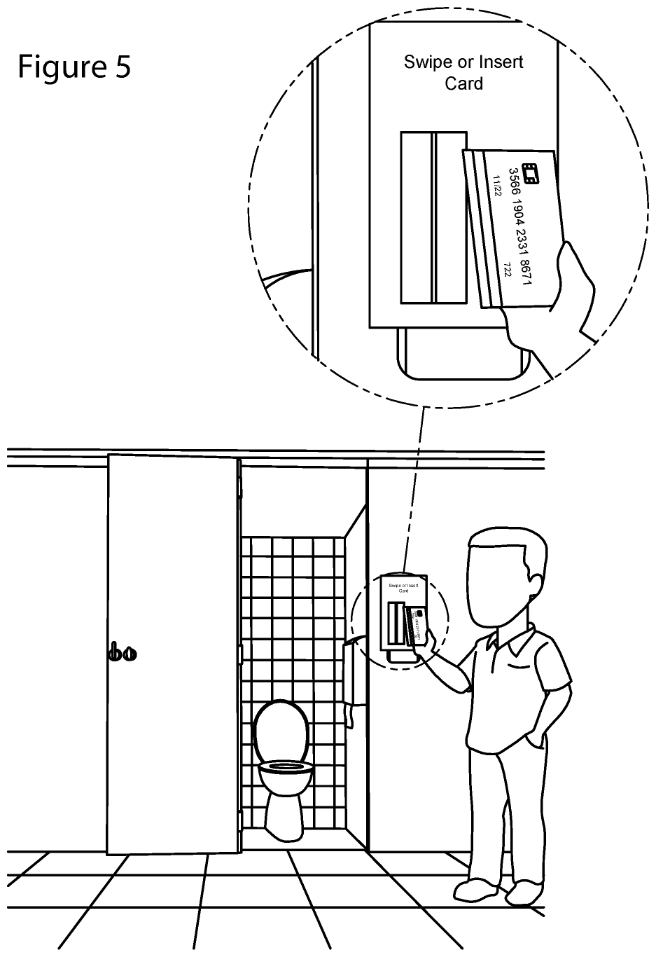

As seen in FIG. 5, an alternative user identification unit works by reading unique magnetic stripes, such as those found on credit cards, for example, as many subway tickets work. The magnetic stripe reader 502 is a part of the user identification unit in FIG. 5. The user is shown to access the station with a membership card 504. An advantage of this user ID unit is that a user can access a station with a physical membership card and would not need a smart phone—an option that might be more appealing to an older population. Preferably, an on-site pre-qualification device prints out temporary cards, or “tickets”, with magnetic stripes, creating a user experience analogous to buying a ticket at a subway station. A physical membership card is not limited to user identification units equipped with magnetic stripe readers, though. QR codes, or similarly optically readable signatures, may be printed upon physical membership cards that are issued to pre-qualified users.

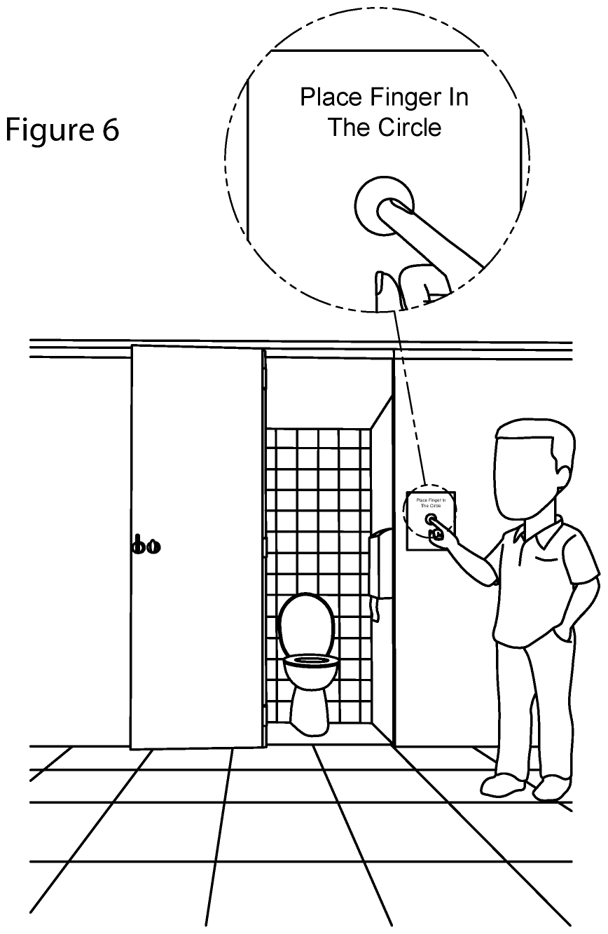

As depicted in FIG. 6, an additional possible user identification unit 602 would employ bioinformatic identification. The user identification unit in FIG. 6 utilizes a finger print scanning device 604 to identify a pre-qualified user. Finger print scanning is not the only bioinformatic scanning identification that may be used. Facial recognition, retinal scanning, and voice print, among others, are possibilities.

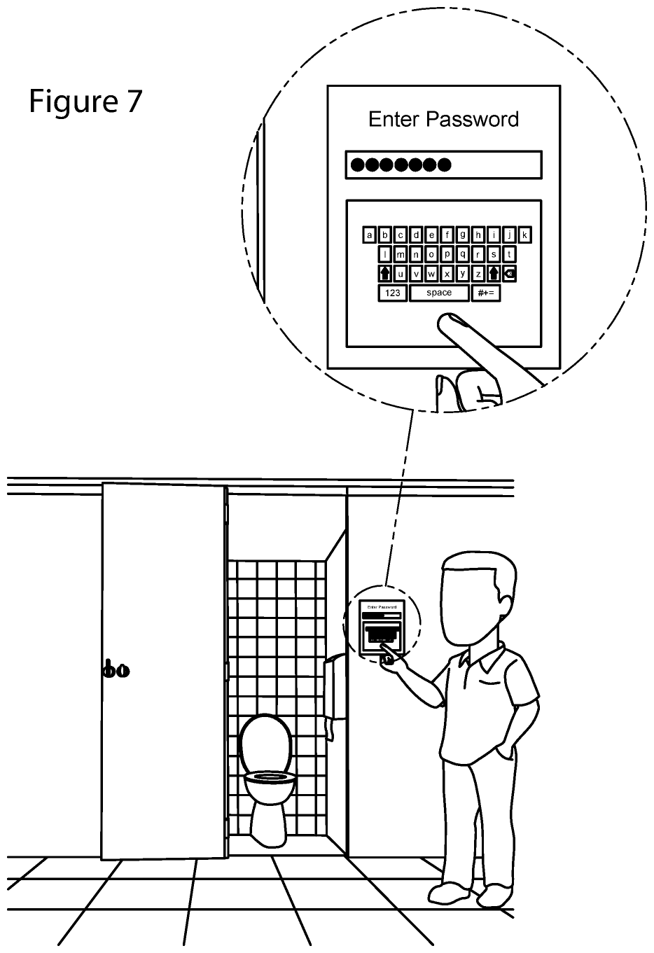

Another method of identification for the user identification unit relies on data entered by the user, i.e. a log-in option requiring a username and password, or full name and unique code, etc. The embodiment depicted in FIG. 7 is an example of a user identification unit 702 that identifies pre-qualified users based on information entered by the user. The information is entered via a keypad on a touchscreen 706. Preferably, the user not only enters identifying information, but a password 704 to verify that they are a pre-qualified individual. A variation of the user identification unit is a touch pad and optical reader embedded within the same user identification unit. This way, users would have the option of producing a QR code, either on a smart device or paper, or using a log-in option from memory, to gain access to the system as a pre-qualified user.

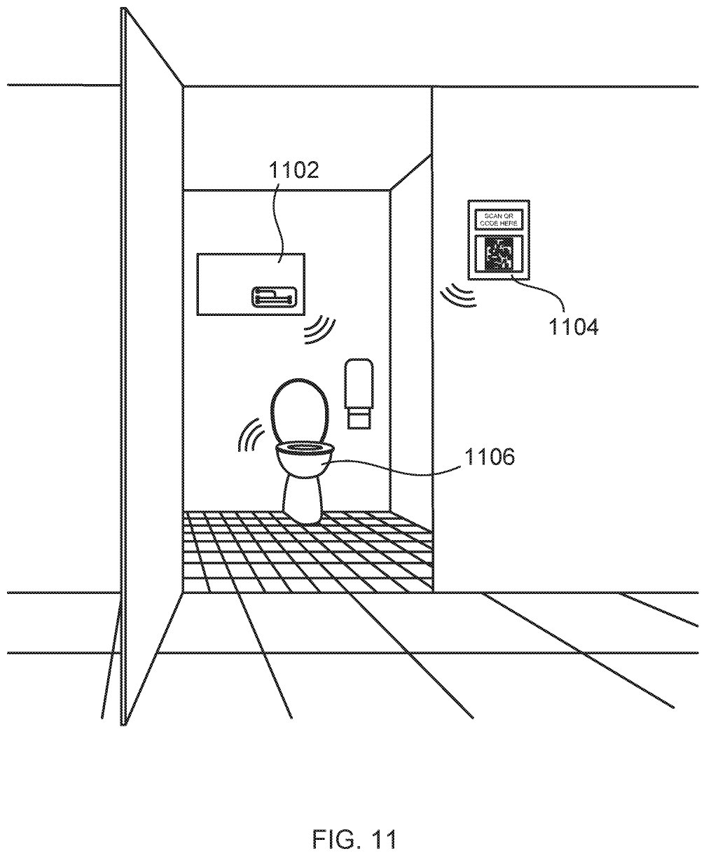

Preferably, the user-identification units have a transmitter and receiver within them, that connects to a central base station as well as a processor within the station. As illustrated in the sample station in FIG. 11, the user identification unit 1104 is able to receive and transmit data to the station processor 1102, as well as an off-site central processor. Information is transmitted from the user identification unit to the central base station, either directly or via the station processor, over the Internet or a localized cloud-based network. In the preferred embodiment, communication between the user identification unit and central base processor is how a user is identified within the pool of pre-qualified users, or if an on-site purchase has been made that qualifies the user access to the station.

Whatever the form of user identification unit, the purpose is not necessarily to lock out users who are not-prequalified. The user identification unit may be used to enable the smart features of the toilet, and identification is necessary to connect the event data to a user profile. However, in the preferred embodiment, the smart toilets may be used and accessed like normal washroom facilities by anyone who chooses not to utilize their other features.

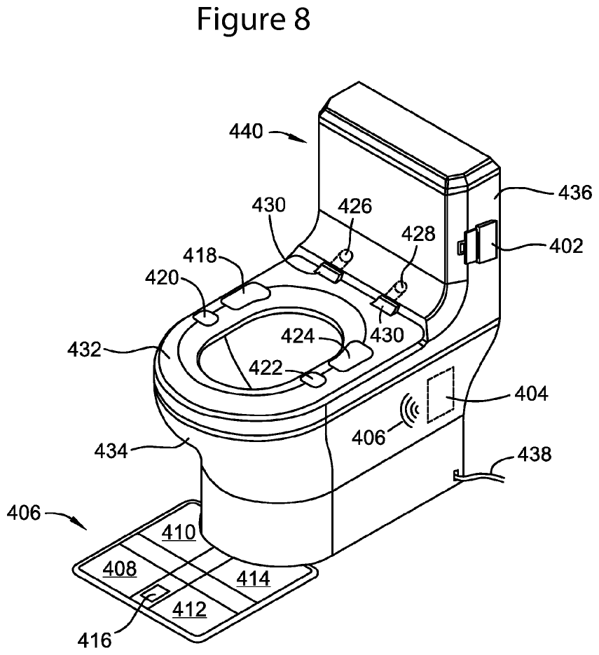

Each station will have a smart toilet. FIG. 8 shows a cross section of a smart toilet. As shown in FIG. 8, the smart toilet has a bowl 802 that receives excreta, and a bowl-clearing mechanism 810 to dispose of the excreta. Additionally, each toilet has two or more sensors that detect properties of the excreta. Sensor 804 and sensor 806 are able to detect certain properties of the excreta. The sensors within the smart toilet transmit signals to station processor 812. The smart toilet can have an attached station processor, as shown in FIG. 8. Alternatively, the smart toilet can have a data communication unit that receives signals from the sensors and transmits the collected data to a processor located within the vicinity of the station. The station processor 812 has a data communication unit that can both receive information from the sensors and send the raw data to a central base processor. Examples of properties that are detected or measured from excreta may include the presence and level of glucose excreted in urine, fat content of fecal excreta, certain hormone levels, certain proteins, and ketones, vitamins present, and presence of blood in urine.

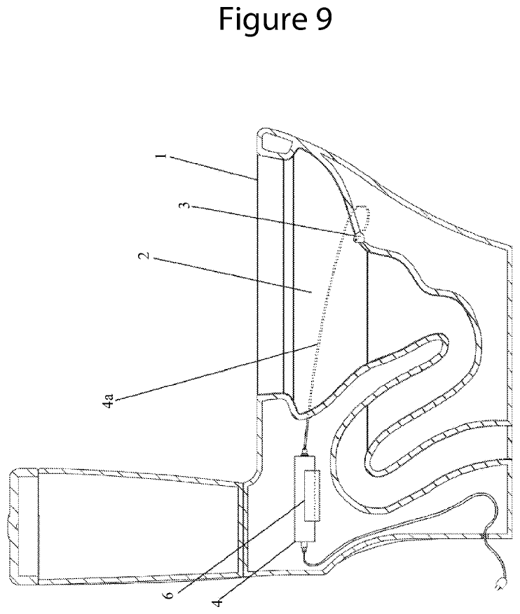

FIG. 8 shows a cross section of the smart toilet, with the bowl, and its apparatus for excreta collection, with sensors for detecting properties of the excreta. FIG. 9 is a top-angle view of a smart toilet that is equipped with additional sensors.

As shown in FIG. 9, the station may also be equipped with sensors external to the toilet to gather other wellness information. A scale 902 measures the user's weight, and sensors 904 and 912 measure the user's blood pressure. In this embodiment, sensor 908 measures the user's temperature, and sensor 906 measures the user's heart rate, also referred to as their pulse. The smart toilet in FIG. 9 has data communication unit 910 through which the smart toilet shares the data collected by the sensors to an external station processor.

Out of respect for the individual user and recognizing the many cultural stigmas surrounding certain health and wellness markers, users may be given the option of which health and wellness properties they would like measured and which ones they would not. For example, many individuals may find it stressful to be weighed.

As seen in FIG. 9, the sensor 902 that detects weight is located on the floor, in front of the toilet. Although it is visible in the drawing, it may be included in the floor without drawing attention to itself. Also seen in FIG. 9, there are various sensors on the toilet seat. Sensor 906 senses heart rate. Sensors 904 and 912, together, measure blood pressure. Preferably, there is also a sensor that measures body temperature. On the smart toilet depicted in FIG. 9, the sensor measuring body temperature 908 is located on the seat.

Other sensors external to the smart toilet may use imaging to measure conditions that can be optically detected, such as blood oxygenation, face sag, leg edema, and skin conditions. This is not a complete listing of the non-excreta sensors that may be added to a smart toilet. The focus of a smart toilet may be the excreta data, but the measurements from these additional sensors, when taken regularly, add significantly to the user's total wellness picture.

While the embodiments of stations shown in FIGS. 1-7 depict one smart toilet in the station, along with one user identification unit and one station processor; in alternative embodiments, the station will include multiple smart toilets. In these alternative embodiments, the station may include a single user identification unit that grants access to the station's multiple smart toilets. For example, the station may have a single access door that is controlled by a single user identification unit. Also, in these alternative embodiments with multiple smart toilets, a single processor may serve all of the smart toilets in the station. In still other embodiments, each of the smart toilets may have its own station processor.

In some embodiments, one or more smart toilets are mixed in with conventional toilets, for example, in a public restroom. In such embodiments, the smart toilets are designated by signage or the like. The smart toilets may also be identified to the pre-qualified user through the app running on his smart phone.

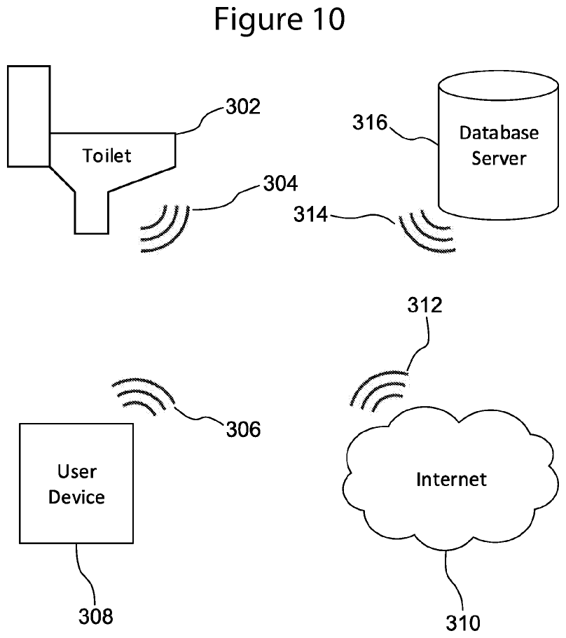

Each station has a processor configured to receive signals from all of the sensors within the station, as well as the user identification unit. FIG. 10 depicts components of the system that are able to send and receive data over the Internet or a localized cloud-based network. Preferably, the station processor 1002 is a single computer, most preferably a System on a Chip. It may be external to the smart toilet but within the station, as the station processor depicted in FIG. 11. Station processor 1102 and the data communication unit receives data from the smart toilet 1106 and user identification unit 1104. The station processor 1102 and the user identification unit 1104 can share data with an off-site central base processor and each other. Another embodiment of the station processor may be physically attached to the smart toilet, as seen in FIG. 9, wherein the station processor 910 may share a hard-wired connection with the sensors. Alternatively, the processor is made up of more than one sub-processor, which are linked to different sensors. Depending on the sophistication of the station processor and the speed and reliability of the data link, it may or may not include data storage at the station level.

The station processor combines the signals from the sensors and the information from the user identification unit 1004 to create unique excreta event data, which is essentially the data that is collected from the sensors at the station and is unique to the user for each station visit. Preferably, the station processor merely collects these data and transmits the raw data to the central base. Alternatively, the station processor may do some processing of the data.

Each station has a station communication data unit, which transmits the excreta event data to the central base data receiving unit 1006, as illustrated in FIG. 10.

The central base is comprised of different components that function together to convert the excreta event data into useful, user-friendly report with health and wellness information. The central base data receiving unit receives the excreta event data from the station communication data unit over the internet. That data is stored at the central base data storage unit. The central base data processor creates reports on the user's health and wellness. The central base data reporting unit is designed to send the information generated by the central base data processor to the appropriate user. This health and wellness report can be received by the user on their smart device, such as the smart phone 1008. Preferably, users will download a designated app to their smart device for receiving and viewing health and wellness reports. Alternatively, a station may include an on-site printer 1010 to print out the generated health and wellness report for the user. The user may also designate an email address to receive the report. These are some examples, but not a complete list of ways in which the central base can send the health and wellness report to the user.

The central base data processor utilizes more data than just the single excreta event data. One purpose of the central base processor's report is to sift through it and make it understandable by the user. When a user pre-qualifies, they are preferably asked health and demographic questions in order to improve the usefulness of the wellness report generated. Although a user will always have the option to disclose information or keep it private; age, gender, and some risk factors may be taken into account when a wellness report is generated by the central base data processor. For example, if a station is equipped with a heart rate monitor, the raw event data would be a number. However, if the user's age is known, the user can be told more precisely how slow or fast their heart is beating relative to its maximum. In cardiovascular fitness and health, percentages of maximum heart rate are more indicative than a heart rate alone. And, when multiple event data are generated, a wellness report is able to differentiate between one isolated low heart rate, or a consistently low heart rate that may indicate an underlying condition. Additionally, if a user inputs known risk factors into their profile, the central base processor can create reports tailored with sensitivities to slight variances to certain measurements. For example, someone at risk of kidney disease might want a lower threshold for an alert for proteins in the blood.

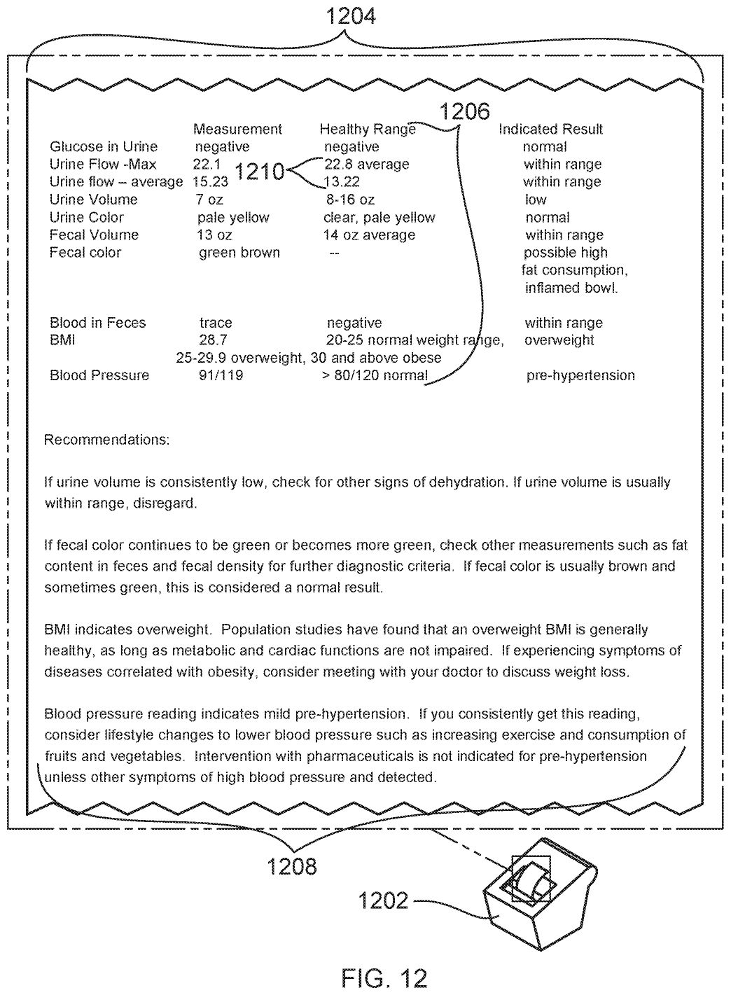

Beyond specific information to the user, norms and trends on all data collected can be included in the event report by the base station. The raw event data is of limited benefit to the user. A feature of this system is the health and wellness reports generated by the central base processor. In the preferred embodiment, these health and wellness reports include not only the measurements taken, but also compare the user's measurements against standard norms. Additionally, the report will indicate if a measurement is “normal” or of possible concern, and it will include additional information to the user about what the findings may indicate and what additional steps should be taken. An example health and wellness report is shown in FIG. 12. The standard norms are communicated to the user as “Healthy Range” 1206. This healthy range, or standard norms, are, for many measurements, not constant for all users, but depend on additional information such as age and sex. As previously mentioned, one method the user may receive the health and wellness report is as a print-out 1204 from on-site printer 1202. In this sample report, the recommendations 1208 provided to the user regarding their blood pressure are dependent on their age. Mild hypertension in younger adults is not known to correlate to cardiovascular risk. However, in elder populations hypertension is more likely to correlate with traumatic health events, such as heart attack and blood clots. So, the recommendations given to the user for their mild hypertension states that pharmaceutical intervention is not indicated, in the absence of other symptoms. This recommendation would be different for a 75-year-old individual. Regarding the standards the user data are measured against, some of them, such as urine flow maximum and average 1210, vary based on sex.

All these data may seem difficult to sift through and make sense of, especially by a user without health and wellness training. Even the sample health and wellness report generated by the central base station can only give users information and recommendations based on one event. A useful tool is the preferred accompanying app, for users with smart device access.

The app may perform many functions and is configured to cooperate with other components of the system. As illustrated in FIG. 10, the app can receive health and wellness reports directly from the central base processor. And, in various embodiments of the user identification unit, the app may be used in tandem with the functions of the user identification unit to access a station. The app may have all of the features listed herein, but it will have a minimum of the specified features.

One feature of the app is that it can identify an individual as a pre-qualified user. The app may also determine if a station is in the network that the user is pre-qualified for, as well as locate stations in the user's network. An app user may input their location, or use the location services embedded in many smart devices, in order for the app to provide a list and location of nearby stations.

As previously disclosed, the app is able to receive and display health and wellness reports. It can also create periodic reports that summarize a user's health and wellness information over a specified time. The app can also create reports about the trends detected in the health and wellness reports. It may present this information to the user in a graph, or a written report. Additionally, the app can compare the user's measured excreta properties to predetermined levels, often referred to as the Healthy Range, as well as to the user's prior levels.

An additional feature of the app is to provide “notifications” to the user. These notifications can be received and displayed in various ways as set by the user. The user may enable the app to create an alert when any of the health and wellness data is outside of a parameter set by the user. Alternatively, the parameters that generate notifications are set by a healthcare provider. The user can enable additional notifications as well. A notification can alert a user that a new health and wellness report has been received, or the app has generated a periodic summary report. Notifications are not necessarily cautionary. The user may configure the app to send them a notification when five consecutive blood pressure measurements are within a desired range, for example. The user may choose to receive notifications as emails, in which case the app will generate an email with the relevant alert.

The app can also generate notifications for individuals beyond the user, such as friends, family members, and healthcare providers. Users may share their notifications with others as a means of sharing their health progress. Alternatively, notifications can be sent to a caretaker, preferably to alert them when information within the health and wellness data indicates the user needs assistance or an adjustment to their current care.

The example health and wellness report in FIG. 12 was created with a single set of excreta event data. The app can provide the user with recommendations to ameliorate or prevent conditions detected in their health and wellness data, drawing from the complete history of their measurements. In the preferred embodiment, the app will provide the user with a plurality of targeted wellness programs to choose from.

There may be different modes for the app, or different apps altogether, depending on the user's goals. A 60-year-old recovering from breast cancer would be interested in different markers than a 33-year-old biohacker. Although health is important to everyone, different modes can provide a better experience for all users. One way to deliver information tailored to the goals of the user is in the form of goal-specific report summaries with accompanying “programs”. One example of a goal-specific program is “Optimal Fitness”. Optimal Fitness, designed for someone who is physically active and trying to improve their physical fitness, gives information about daily hydration, metabolic markers, and heart rate at the top. The recommendations associated with the report can be specialized to this goal, as well. For example, athletes who exercise for an hour or longer are advised to increase the amount of water they drink. The report will indicate that a user may be under-hydrating, based on their urine flow, for their goals, even if the urine flow falls within otherwise normal ranges. Short fitness tips or short exercise videos can be included as well.

Another targeted wellness program is “Heart Health”, designed for someone who is monitoring or improving their cardiac health. This program is designed for someone with an active interest in minimizing their risk of cardiovascular disease, whether due to heredity factors, age, or existing conditions. The Heart Health program shares daily sodium, urine outflow, and blood pressure. In addition to highlighting the relevant data, the Heart Health program provides the user with in-app suggestions and videos targeted to them, such as five-minute mindfulness exercises, cooking tips for minimizing sodium consumption, or a daily motivating reminder to go for a walk.

The programs can be designed to incentivize users to improve their off-target measurements or meet their goals. For example, every time a blood pressure is measured lower than the user's average, some form of “points” can be awarded. Alternatively, the user may receive a notification when their blood pressure has decreased saying, “Great job, your blood pressure is lowering! Keep up the good work.” A user can opt in to as many of these targeted programs as they wish.

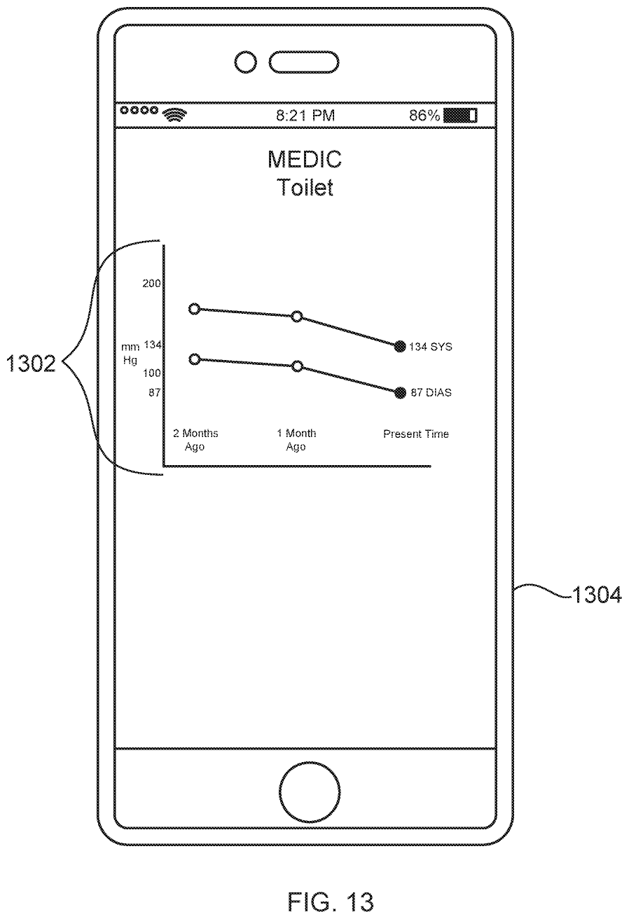

FIG. 13 shows one screenshot of the app's interface. Trends for health measurements can be viewed graphically, such as graph 1302. This is a graphic depiction of the user's blood pressure over a two-month period. A benefit of viewing some health and wellness measurements on a graph is that trends in data are easy for the user to note. An additional feature of the app is that the user can adjust their app settings so that their smart device alerts them if any out-of-range or pre-specified measurement is detected, and otherwise can have peace of mind knowing their hydration levels and blood pressure are healthy.

While it is expected that most users will elect to receive a report after each excreta event, other users may elect to receive reports less frequently, for example, weekly or monthly. Also, some users may elect to receive period summaries, in the form of weekly or monthly reports, in addition to reports after each excreta event.

While the primary audience for the health and wellness report is the individual user, the report may also be shared with others. For example, a user may choose to share the reports, or at least some of the data therein, with a healthcare provider, e.g. a physician or health maintenance organization. In this way, the healthcare provider is regularly advised of health and wellness indicators and may even choose to intervene and contact the user if adverse indicators are detected.

In another embodiment, the reports, or at least a portion thereof, are shared with a medical services group, an insurance company or Health Maintenance Organization (HMO). In this way, the group, company or organization would likely reduce the expenses by monitoring the health and wellness of members of that group and providing or at least suggesting preventative measures.

Naturally, all systems of the present invention need to be designed with security and privacy measures to comply with state and federal laws and regulations, for example the Health Insurance Portability and Accountability Act (HIPPA) and the Patient Protection and Affordable Care Act (PPACA). The systems should also comport with users' expectations of privacy.

In yet another embodiment, the user may opt to share the reports with family members or friends.

While, in the preferred embodiment, the reports are delivered digitally to the users through an app on a smart phone, tablet or computer; the reports may also be delivered by other digital means such as an e-mail or text message. Alternatively, especially for the embodiment where the user is using an on-site pre-qualification mechanism, i.e. “one off” registration, the report may be delivered by printing a paper or card with the report for the user to take away, as seen in FIG. 12.

As mentioned, there are different demographics served by the smart toilet. One popular setting may be the fitness center, as people who regularly visit a gym are likely more interested in wellness information than those who do not. A large, multi-location chain of fitness centers may buy or lease several smart toilets and offer access to their network of stations to their members as a premium, monthly add-on. Members of the gym chain who paid the add-on would then be pre-qualified users for every station in every gym of that chain. A different option would be for a high-end gym to provide smart toilets at all of their locations as a standard feature. An individual would still need to go through the process of pre-qualification, i.e. creating an account, acquiring a unique user ID for the identification units. Nevertheless, in this case, the subscription price would be underwritten entirely by the gym.

Another setting where a network of smart toilets can provide great benefit is healthcare facilities. As residences for the disabled and elderly, care homes are filled with people at risk of nutritional deficiency, medication mix-ups, and many illnesses that increase in incidence with age. Often, residents of care homes have many of their health markers monitored regularly. A network of smart toilets in a care home can save time for staff and discomfort for its residents. The presence of smart toilets, in providing physical and mental comfort to residents and their family members, would make one care home more appealing than another, and thus incentivizing care homes to provide them as part of their comforts and conveniences.

There are primarily two different modalities for the network of smart toilets. Although every unit is a public toilet in the sense that it is not in a private home, some toilets are not accessible to everyone. For example, when the system uses stations located within a given fitness club or group of fitness clubs, only members of that fitness club have access to those stations. The same would be true for stations located within a place of employment, a care facility or other private space. In at least some of these embodiments, all of the individuals within the private space are also qualified users, such as a premium gym chain that provides access to all of its members. Nevertheless, even in those embodiments, each user must be identified within the system, so that the health and wellness data are linked to that user.

In other embodiments, the system may include stations located in a variety of public spaces, such as public parks, transportation centers, retail malls, public buildings, such as civic halls, etc. In these other embodiments, it is preferable that each of the stations are clearly marked as belonging to the health and wellness monitoring network. In this way, a user that is pre-qualified, for example by having purchased a subscription, can find the stations within the network and take advantage of the benefits of using that station within the network. One feature of the app is an option to find the nearest station to you, based on location services.

In still other embodiments, stations within the same network are located in both private and public spaces. Ideally, the users can find in-network stations at their place of employment, their fitness club and in public spaces, such as airports, shopping malls and the like. When all of these stations are within the same network, the user is able to obtain health and wellness data wherever they can find a station.

Although much has been described of users being pre-qualified by belonging to a particular group, or by purchasing a subscription, in still other embodiments, a user can become pre-qualified on-the-spot. In this embodiment, a potential user merely approaches the station and follows instructions to pay for the service provided by the smart toilet. Also, with this on-the-spot registration, the user is given a choice of downloading an app to receive a report, of receiving the app by text or e-mail, or of having that report printed and delivered at the station.

This invention provides a system of how a network of smart toilets can be distributed and networked throughout the public. As such, one method of pre-qualification may be an individual choosing to subscribe. A user may become pre-qualified through a dedicated app, or by accessing a subscription network online. Subscription options may be for a certain time period, e.g. one month, and renewable, or they may be twenty visits, renewable. They may be some combination, such as a maximum number of ten visits a month.

Publicly-accessibly stations may be distributed by public or private entities. For example, a municipal parks department may see an opportunity to raise money for their department and erect a station at the city's most popular park, and purchase a license connecting it to a larger network. Alternatively, that same parks department can lease the space for the same purpose and the station would be on a public space, but this time run by a private entity. Another embodiment for the network is a city choosing to establish multiple stations throughout its public parks system. An entrepreneur may run a chain of stations out of shopping centers and movie theatres, leasing the space from private companies.

Different systems of stations may all share certain connectivity features, although this is up to the purveyors. In the preferred embodiment, a pre-qualified user within one network will be able to integrate health and wellness data from out-of-network stations into their profile, even if they are required to make a one-off purchase to use the out-of-network toilet. Another feature of the preferred embodiment is that different networks, such as one established in healthcare facilities and one established in a city's public parks, will have cost-sharing or open access agreements between each other, in order to widen the network and increase accessibility.

Additionally, in the case that a user owns a smart toilet at their home, they will preferably be able to integrate the data from their private smart toilet with the other network toilets so that their health and wellness reports include all data.

Another entity that may be interested in providing access to the stations is insurance providers. Since insurance providers usually do not have their own space that consumers regularly visit, they can provide the type of membership that would apply to a publicly-accessibly network. Or, as in the HMO model, a network of healthcare providers may all provide their patients with subscriptions and their providers with stations.

Although repeated reference has been made to “subscriptions”, with the alternative of an on-site one-time payment, a user may register an account but still pay on a per-visit basis.

It is noted that all patents and published patent applications referred to herein are incorporated herein by reference.

The invention has been described with reference to various specific and preferred embodiments and techniques. Nevertheless, it is understood that many variations and modifications may be made while remaining within the spirit and scope of the invention.

What is claimed is:

1. A system for monitoring and reporting health and wellness data comprising:
a pre-qualification mechanism, whereby an individual can become a pre-qualified user;
a plurality of stations, each station comprising:
a user identification unit to determine if the user is a pre-qualified user and, if so, identify that pre-qualified user;
a toilet comprising
a bowl to receive excreta;
two or more sensors configured to detect properties of the excreta from
a pre-qualified user; and
a bowl clearing mechanism to dispose of the excreta;
a station processor configured to receive signals from the two or more sensors and from the user identification unit to create excreta event data for the identified pre-qualified user;
a station data communication unit;
a central base comprising:
a central base data receiving unit configured to receive excreta event data via the station data communication unit from each of the plurality of stations;
a central base data storage unit configured to store excreta event data;
a central base data processor configured to process the excreta event data for each pre-qualified user and produce health and wellness data for that pre-qualified user;
a central base data reporting unit configured to report health and wellness data to the appropriate pre-qualified user.

2. The system of claim 1, wherein the pre-qualification mechanism involves group membership, whereby individuals become pre-qualified users by being members of a particular group.

3. The system of claim 2, wherein the particular group is selected from patients of a healthcare practice, members of a fitness club, employees of a company, individuals insured by a certain provider, and residents of a care facility.

4. The system of claim 1, wherein the user identification unit employs a signal transmitted from the user's smart device.

5. The system of claim 1, wherein the user identification unit scans an optically readable code.

6. The system of claim 1, wherein the user identification unit scans a magnetically readable code.

7. The system of claim 1, wherein the user identification unit employs bioinformatic identification.

8. The system of claim 1, wherein the user identification unit employs an optically readable code, which is scanned by the user's smart device and transmitted to the central base, which, in turn, transmits a signal to the user identification unit of the station indicating that the user is pre-qualified.

9. The system of claim 1, wherein the pre-qualification mechanism is configured for a user to register with the system on-the-spot.

10. The system of claim 9, wherein the user purchases an on-the-spot registration.

11. The system of claim 1, wherein the pre-qualification mechanism involves a paid subscription by the user.

12. The system of claim 11, wherein the paid subscription provides access for the user to all of the stations in the system, regardless of location.

13. The system of claim 1, wherein the properties detected from the excreta are selected from the group consisting of; level of glucose in urine; level of protein in urine; identification of proteins in urine; level of various salts in urine; urine flow; urine volume; urine color; urine temperature; fecal volume; fecal density; fecal color; fecal temperature; gas identification and volumes; blood in feces and combinations thereof.

14. The system of claim 1 wherein the station further comprises additional sensors that measure non-excreta properties of the pre-qualified user and wherein those non-excreta properties are used by the central base processor to produce the health and wellness data.

15. The system of claim 14, wherein the health and wellness data other than from the excreta are selected from the group consisting of body temperature, body weight, pulse rate, blood pressure, skin color, skin conditions and combinations thereof.

16. The system of claim 1, wherein the central base data processor is configured to identify trends in users' health and wellness data.

17. The system of claim 1, wherein the pre-qualified user receives reports on the health and wellness data digitally.

18. The system of claim 1, wherein the system further comprises an app running on the user's smart device configured to cooperate with other parts of the system to perform at least two of the following functions:
identifying the user as a pre-qualified user;
identifying the station as in-network;
locating an in-network station;
receiving and displaying reports based on the health and wellness data;
creating periodic reports summarizing the health and wellness data;
creating reports comprising trends detected in the health and wellness data;
comparing measured properties of the excreta with predetermined levels;
comparing properties of the excreta with prior levels for the user;
receiving a notification when any of any of the health and wellness data is outside a parameter set by the user;
receiving a notification when any of the health and wellness data are outside a parameter set by the user's healthcare provider;
sending a notification to at least one individual, pre-selected from family members, friends and healthcare providers, when any of the health and wellness data are outside a pre-set parameter;
providing the user with instructions to ameliorate or prevent a condition detected from the health and wellness data; and
providing the user with a plurality of reporting options, depending on the user's health and wellness goals.

19. The system of claim 1, wherein the station is configured to allow users who are not pre-qualified to access the toilet function of the station.

20. The system of claim 1, wherein the health and wellness data reported to the pre-qualified user is printed at the station and delivered to the pre-qualified user.
